(19) United States
(12) Patent Application Publication
(10) Pub. No.: US 2007/0157323 A1
(43) Pub. Date: Jul. 5, 2007

(54) In vivo odorant receptor systems and their uses

(76) Inventors: John R Carlson, North Haven, CT (US); Elissa A. Hallem, Arcadia, CA (US)

Correspondence Address: COOLEY GODWARD KRONISH LLP;ATTN: Patent Group, Suite 500, 1200 - 19th Street, NW, WASHINGTON, DC, 20036-2402, US

(21) Appl. No.: 10/585,950

(22) PCT Filed: Dec. 20, 2004
(86) PCT No.: PCT/US04/42372

§ 371 Date: Jan. 10, 2007

Related U.S. Application Data
(60) Provisional application No. 60/531,113, filed on Dec. 20, 2003.

(51) Int. Cl. A01K 67/033; G01N 33/567; C12N 5/06

(52) U.S. Cl. 800/3; 435/7.2; 800/13; 435/348

(57) ABSTRACT

The present invention provides in vivo materials, systems and methods for the reliable and efficient screening of odorant receptors (ORs), particularly ORs isolated from insects.

### RELATED APPLICATIONS

This application claim s priority to U.S. provisional patent application Ser. No. 60/531,113 filed Dec. 20, 2003 which is herein incorporated by reference in its entirety.

### STATEMENT REGARDING FEDERALLY SUPPORTED RESEARCH OR DEVELOPMENT

This invention was supported in part by funds obtained from the U.S. Government (National Institutes of Health Grant Numbers DC-02174 and DC-04729) and the U.S. Government may therefore have certain rights in the invention.

### BACKGROUND OF THE INVENTION

Animals are able to sense and discriminate among a remarkable number of odors. Olfactory information is received and encoded by olfactory receptor neurons (ORNs). These neurons encode the quality and intensity of odors, as well as aspects of their spatiotemporal distribution. The code is in the form of action potentials and is based on the differential responses of ORNs to different olfactory stimuli. The signals generated by ORNs are transmitted to the brain, where processing takes place (Dobritsa, 2003). ORNs vary in their odor specificity, sensitivity, and response dynamics. Olfaction plays a critical role in many insect behaviors, including identification of food, mates, and predators (Takken, 1991; Hildebrand and Shepherd, 1997).

Olfactory signal transduction is widely conserved across a broad spectrum of organisms, including mammals, fish, crustaceans, nematodes, and insects (Hildebrand and Shepherd, 1997).

The olfactory signaling cascade is initiated by G-protein-coupled receptors (GPCRs). GPCRs are a sequence-diverse group of integral membrane proteins with seven hydrophobic domains; they are therefore called Seven-Transmembrane or Serpentine Proteins. Their main role is the transmission of signals from the outside of the cell (typically a nerve cell or neuron) to the inside, via interaction with an external agonist or antagonist and an internal protein/ion/effector pathway involving trimeric G proteins such as G.alpha (U.S. Patent Publication No. 20030165879). GPCRs involved in olfactory signaling cascade are known as odorant receptors (ORs) (Buck and Axel, 1991; Ngai et al., 1993). Odorants bind either directly or indirectly to these odorant receptors and activate specific G-proteins. The G-proteins then initiate a cascade of intracellular signaling events leading to the generation of an action potential which is propagated along the olfactory sensory axon to the brain (U.S. Patent Publication No. 20020064817; Mombaerts, 1999; Pilpel et al., 1998).

A large multigene family thought to encode odorant receptors was initially identified in the rat (Buck and Axel, 1991). The first invertebrate organism in which candidate ORs were identified was Caenorhabditis elegans through the screening of a genome project for potential signaling molecules (Troemel et al., 1995). Using a variety of approaches, a large family of candidate ORs was recently identified in D. melanogaster (Gao and Chess, 1999; Clyne et al., 1999; Vosshall et al., 1999). Furthermore, several studies have used a variety of methods to begin to examine OR-odorant interactions (Zhang et al., 1997; Zhao et al., 1998; Wetzel et al., 1999; Storkuhl and Kettler, 2001; Wetzel et al., 2001).

### SUMMARY OF THE INVENTION

The present invention provides materials, systems and methods for the reliable and efficient screening of odorant receptors (ORs) from animals, particular from insects. The present invention provides materials, systems and methods to access the ability of various molecules to bind with ORs, as well as to access the strength of such binding when it occurs. Also, the present invention provides materials, systems and methods for screening the ability of different molecules to reduce, inhibit or enhance the binding of the ligand molecules which would naturally or normally bind to ORs.

### BRIEF DESCRIPTION OF THE DRAWINGS

FIG. 1. Odor-response spectrum conferred by AgOr1 (a) and AgOr2 (b) on a Drosophila olfactory neuron carrying a deletion of its endogenous receptor genes Or22a and Or22b (response of deletion mutant without transgenes is shown in c). N=12; error bars=Standard Error of the Mean (SEM).

### DETAILED DESCRIPTION OF THE INVENTION

All publications and patent applications herein are incorporated by reference to the same extent as if each individual publication or patent application was specifically and individually indicated to be incorporated by reference.

The following description includes information that may be useful in understanding the present invention. It is not an admission that any of the information provided herein is prior art or relevant to the presently claimed inventions, or that any publication specifically or implicitly referenced is prior art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

### I. DEFINITIONS

As used herein, the term “allomones” refers to any chemical substance produced or acquired by an organism that, when it contacts an individual of another species, evokes in the receiver a behavioral or developmental reaction adaptively favorable to the transmitter.

As used herein, the term “animal” refers to any multicellular organism of the kingdom Animalia.

As used herein, the term “host” refers to any organism on which another organism depends for some life function. Examples of hosts include, but are not limited to, humans which may serve as a host for the feeding of certain species of mosquito and the leaves of soybeans (Glycine max(L.)) which may act as hosts for the oviposit of the green cloverworm (Plathypena scabra (F.)).

As used herein, the term “kairomones” refers to any of a heterogeneous group of chemical messengers that are emitted by organisms of one species but benefit members of another species. Examples include, but are not limited to, attractants, phagostimulants, and other substances that mediate the positive responses of, for example, predators to their prey, herbivores to their food plants, and parasites to their hosts. Kairomones suitable for the purposes of the invention and methods of obtaining them are described, for example, Science (1966) 154, 1392-93; Hedin, (1985) Bioregulators for Pest Control, American Chemical Society, Washington, 353-366.

As used herein, the term “ligand” refers to a molecule that binds to a receptor or a protein. Ligands may be agonists or antagonists.

As used herein, olfactory receptor neurons (ORNs) refers to the neurons which receive and encode olfactory information. These neurons encode the quality and intensity of odors, as well as aspects of their spatiotemporal distribution. The code is in the form of action potentials and is based on the differential responses of ORNs to different olfactory stimuli. The signals generated by ORNs are transmitted to the brain, where processing takes place. ORNs vary in their odor specificity, sensitivity, and response dynamics. Individual receptors can be mapped to individual neuronal classes through a genetic and molecular analysis. For example, the Or22a receptor of Drosophila has been mapped to the ab3A neuron, by using its promoter and the GAL4-UAS system (Brand et al. (1993) Development 118:401-415).

As used herein, the term “pheromone” refers to a substance, or characteristic mixture of substances, that is secreted and released by an organism and detected by a second organism of the same or a closely related species, in which it causes a specific reaction, such as a definite behavioral reaction or a developmental process. Examples include, but are not limited to, the mating pheromones of fungi and insects. More than a thousand moth sex pheromones (Toth et al., (1992) J. Chem. Ecol. 18, 13-25; Arn et al., (1998) Appl. Entomol. Zoo. 33, 507-511) and hundreds of other pheromones have now been identified, including aggregation pheromones from beetles and other groups of insects. Various compositions, including resins and composite polymer dispensers, have been developed for the controlled release of pheromones have been developed (see, e.g., U.S. Pat. Nos. 5,750,129 & 5,504,142).

As used herein, “protein” means any peptide-linked chain of amino acids, regardless of length or post-translational modification, e.g., glycosylation or phosphorylation.

As used herein, the term “receptor” refers to a binding site located on a cell's surface or interior that responds to a specific molecule of substance.

As used herein, the term “semiochemical” refers to any chemical substance that delivers a message or signal from one organism to another. Examples of such chemicals include, but are not limited to, pheromones, kairomones, oviposition deterrents, or stimulants, and a wide range of other classes of chemicals (see, for example, Nordlund, (1981) Semiochemicals: A review of the terminology, in: Nordlund et al., (ed.) Semiochemicals: Their Role in Pest Control, John Wiley; Howse et al., (1998) Insect Pheromones and Their Use in Pest Management, Chapman & Hall, London).

As used herein, the term “volatile” refers to a chemical which evaporates readily at those temperatures and pressures which are considered the relevant temperatures and pressures for the reference organism of interest. Thus, the phrase “volatile chemical” refers to a chemical that can exist in vapor form and be carried in the air, such as alcohols, aldehydes, acetate esters, organic acids, ketones, and terpanes. Volatile chemicals include natural chemicals and those made by humans.

### II. INSECTS AND THEIR USES IN THE PRESENT INVENTION

The present invention is used for studying and identifying odorant receptors from any insect species, but finds it greatest usefulness for studying the odorant receptors from both beneficial insects and insect pests. The identification of specific odorant receptors that bind with particular chemicals permits screening for ligands that activate or inhibit these receptors and/or modifying the structure of the ligands to alter their binding specificity. Thus, the materials, systems and methods of the present invention are useful for identifying specific chemicals that bind specific odorant receptors and using this knowledge to select and/or design chemicals useful for attracting or repelling the insects.

Thus, the materials, systems and methods of the present invention are advantageously employed for combating, controlling, or attracting one or more insect species of choice. Representative insect species that could be utilized in the odorant receptor systems of the present invention are listed below.

Fruit (including citrus), nut, and vine crops are susceptible to attack by a variety of pests, including sphinx moth larvae, cutworms, skippers, fireworms, leafrollers, cankerworms, fruitworms, girdlers, webworms, leaffolders, skeletonizers, shuckworms, hornworms, loopers, orangeworms, tortrix, twig borers, casebearers, spanworms, budworms, budmoths, and a variety of caterpillars and armyworms.

Field crops are targets for infestation by insects including armyworm, asian and other corn borers, a variety of moth and caterpillar larvae, bollworms, loopers, rootworms, leaf perforators, cloverworms, headworms, cabbageworms, leafrollers, podworms, cutworms, budworms, hornworms, and the like. Pests also frequently feed upon bedding plants, flowers, ornamentals, vegetables, container stock, forests, fruit, ornamental, shrubs and other nursery stock. Even turf grasses are attacked by a variety of pests including armyworms and sod webworms.

The chrysomelid genera Diabrotica and Acalymma contain numerous pest species, including the western corn rootworm (WCR), Diabrotica virgifera virgifera LeConte; the southern corn rootworm (SCR) or, the spotted cucumber beetle, D. undeciltpunctata howardi [D. duodecimpunctate Fab.]; the northern corn rootworm (NCR), D. barberi Smith and Lawerence; and the striped cucumber beetle (SCB), Acalymma vittatum (Fabr.).

Mosquitoes are the most dangerous animals in the world, killing an estimated two to three million people per year. In the United States alone, there are 150 different species. Each species carries different types of diseases and will typically breed and feed at different times of the day. The most common species found in the U.S. include the Aedes albopictus, Culex pipitens and Anopheles quadrimaculatus.

Malaria is one of the leading causes of illness and death in the world. Approximately 300 million people worldwide are affected by malaria and between 1 and 1.5 million people die from it every year. Malaria is caused by a parasite (Plasmodium) that lives in red blood cells and cells of the liver. The parasite is transmitted from person to person by the bite of an infected female Anopheles mosquito. The high vectorial capacity of these mosquitoes is especially due to their strong preference for human hosts, which they locate through olfactory cues. Many aspects of mosquito behavior, including host location, are mediated by detection of volatile semiochemicals (Pickett and Woodcock, 1996; Gibson and Torr, 1999; Takkern and Knols, 1999). They generally show a marked attraction to carbon dioxide, sweat and other olfactory cues emanating from the host. The exact role and chemical nature of these cues remain largely undefined and the molecular mechanisms underlying this process are unknown (Takken, 1996).

Representative insects which can be studied using the materials, systems and methods of the present invention include but are not limited to Delphacidae sp., such as Nilaparvata lugens, Nilaparvata oryzae and Sogatella furcifera; and/or Cicadellidae sp., e.g. Empoasca decipiens, Nephotettix apicalisi, Nephotettix impicticeps, Nephotettix cincticeps and Nilaparvata oryzae; and/or Pyralidae sp., e.g. Tryporyza incertulas, Tryporyza innotata, Cnaphalocrosis medinalis, Chilo loftini, Chilo suppressalis, Chilo indicus and Chilotraea plejadellus; Tylenchidae sp., e.g. Ditylenchus dipsaci, Ditylenchus angustus and Ditylenchus radicicolus; and/or Noctuidae sp., e.g. Sesainia interens, Sesainia calamistis and Sesamnia cretica; and/or Pentatomidae sp., e.g. Scotinophara lurida and Scotinophara coarctata; and/or Plutellidae sp., e.g. Plutella xylostella; and/or Tortricidae sp., e.g. Archips breviplicanus; and/or Cecidomnyiidae sp., e.g. Orselia oryzae and Pachydiplosis oryzae.

Representative soil borne insects include but are not limited to Aeneolamia sp., Agrotis sp. ,e.g. Agriotes sp., Araecerus sp., Aulacophora sp., Atherigona sp., Cerotoma sp., Chilo sp., Cylas sp., Delia sp., Diabrotica sp., Diaprepes sp., Elasmnopalpus sp., Frankliniella sp., Graphognathus sp., Gryllotalpa sp., Hypomneces sp., Heteronychus sp., Holotrichia sp., Hydraecia sp., Hylemia sp., Leucopholis sp., Lepidiota sp., Limonius sp., Listroderes sp., Loxostege sp., Mamestra sp., Melolontha sp., Oscinella sp., Ostrinia sp., Otiorhynchus sp., Phyllophaga sp., Phyllotreta sp., Popillia sp., Pseudococcus sp., Psila sp., Psylloides sp., Sitona sp., Spoladea sp., Tanymecuis sp., Thrips and Tribolium sp.

Representative lepidopteran insects include but are not limited to Pectinophora gossypiella, Bupalus piniarius, Cheimatobia brumnata, Lithocolletis blanicardella, Hyponoineuta padella, Plutella sp., e.g. Plutella xylostella, Malacosomia neustria, Euproctis chrysorrhoea, Lyinantria sp., e.g. Bucculatrix thurberiella, Phyllocnistis citrella, Agrotis sp., e.g. Agrotis segetum, Agrotis ipsilon, Euxoa sp., Feltia sp., Earias insulana, Heiothis sp., e.g. Helicoverpa arinigera, Helicoverpa armigera, Helicoverpa zea, Laphygma exigua, Mamestra brassicae, Panolisflamimea, Prodenia litura, Spodoptera sp., e.g. Spodoptera littoralis, Spodoptera litura, Spodoptera exigua, Trichoplusia ni, Cydia pomonella, Pieris sp., Chilo sp., e.g. Chilo suppressalis, Pyrausta nubilalis, Ephestia kuehiziella, Galleria mellonella, Cacoecia podana, Capua reticulana, Choristoneura fumiferana, Clysia ambiguella, Hofinannophila pseudospretella, Homnona magnanima, Tineola bisselliella, Tinea pellionella, Elasinopalpus sp., Hydraecia sp., Loxostege sp., Ostrinia sp., and Spoladea sp., e.g. Tortrix viridana.

Representative coleopteran insects include but are not limited to Anobium punctatum, Rhizopertha dominica, Bruchidius obtectus, Acanthoscelides obtectus, Hylotrupes bajulus, Agelastica alni, Leptinotarsa deceinlineata, Phaedon cochleariae, Diabrotica sp., e.g. Diabrotica undecimpunctata, Diabrotica virgifera, Psylloides chiysocephala, Epilachna varivestis, Atomnaria sp., e.g. Atomnaria linearis, Oiyzaephilus suriltamensis, Anthonomus sp., e.g. Anthonomus grandis, Otiorhynchus sulcatus, Cosinopolites sordidus, Ceuthorrynchus assimils, Hypera postica, Dermestes sp., Trogoderma sp., Anthrenus sp., Attagenus sp., Lyctus sp., e.g. Meligethes aeneus, Ptinus sp., e.g. Niptus hololeucus, Gibbium psylloides, Triboliun sp., e.g. Tenebrio molitor, Agriotes sp., e.g. Agriotes lineatus, Conoderus sp., e.g. Melolontha melolontha, Amphimallon solstitialis, Aeolus sp., Araecerus sp., Aulacophora sp., Cerotoma sp., Chaetocneima sp., Cylas sp., Diaprepes sp., Graphognathus sp., Heteronychus sp., Holotrichia sp., Hyponmeces sp., Leucopholis sp., Lepidiota sp., Limonius sp., Listroderes sp., Melanotus sp., Phyllotreta sp., Phyllophaga sp., Popillia sp., Sitona sp., and Tanymnecus sp., e.g. Costelytra zealandica.

Representative dipteran insects include but are not limited to Drosophila melanogaster, Chrysomyxa sp., Hypoderma sp., Tannia sp., Bibio hortulanus, Oscinella frit, Phorbia sp., Pegomyia hyoscyami, Ceratitis capitata, Dacus oleae, Tipula sp., Tipula paludosa, Atherigona sp., Delia sp., and Hylemnia sp., Psila rosae, and Tipula oleracea.

There are 10,0000-20,000 species of bee including many wasplike and flylike bees. Most bees are small from 2 mm (0.08 inches) long to 4 cm (1.6 inches) long. Bees and wasps are closely related. Examples of common bees are paper wasps (Polistes, multiple species), yellow jackets (Vespula, multiple species), baldfaced hornets (Vespula) bumble bees (Bomzbus, multiple species), honeybees (Apis mellifera), small carpenter bees (Ceratina, multiple species) and large carpenter bees (Xylocopa, multiple species).

### III. VOLATILE AND SEMI-VOLATILE CHEMICALS

Volatile and semi-volatile chemicals are emitted from all living organisms either naturally or following a particular interaction with their environment (e.g., attack by another organism, wind, rain, hail, change in light intensity, etc.). Representative examples of such volatile and semi-volatile chemicals are well known to those skilled in the art and include, but are not limited to the following: catnip scent, terpenoids, indole, cinnamaldehyde, cinnamyl alcohol, phenethyl proprionate, eugenol, geraniol, methyleugenol, 2-allyl-6-methoxyphenol (orthoeugenol), paradimethoxybenzene, veratrole (1,2-dimethoxybenzene), phenylacetaldehyde, chavicol (4-hydroxy-1-allylbenzene), beta.-ionone; 4-methoxycinnamaldehyde; 4-methoxycinnamonitrile; 4-methoxy-1-vinyl-benzene; 4-methoxy-1-propyl-benzene; 4-methoxy phenyl ethyl ether; 4-methoxy phenyl acetonitrile; allyl benzene; cinnamonitrile; 2-methoxy cinnamaldehyde; cinnamyl acetate; cinnamic acid methyl ester; dihydrocinnamyl aldehyde; phenyl propionitrile, 4-methylphenylethanol, 4-chlorophenylethanol, 4-fluorophenylethanol, phenylethanol, phenylpropanol, 4-methoxyphenylethanol, 3-methoxyphenlyethanol, 2-methoxyphenylethanol, 4-methoxyphenylpropanol, and phenyletliylamine and phenylpropylamine.

Some of these volatile and semi-volatile chemicals play a role in attracting or repelling animals, including insects. Herbivore attack is known to increase the emission of volatiles, which attract predators to herbivore-damaged plants in the laboratory and agricultural systems.

Plant leaves emit a broad spectrum of organic compounds that typically play multiple roles in plant protection (Niinemets et al., Trends Plant Sci. April 2004; 9(4):180-6). For example, green leafy volatiles (GLV), six-carbon aldehydes, alcohols, and esters commonly emitted by plants in response to mechanical damage or herbivory, induced intact undamaged corn seedlings to rapidly produce jasmonic acid (JA) and emit sesquiterpenes (Engelberth et al., Proc Natl Acad Sci USA. Feb. 10, 2004;101(6):1781-5).

Kessler et al. (Science. Mar. 16, 2001; 291(5511):2141-4) quantified volatile emissions from Nicotiana attenuata plants growing in natural populations during attack by three species of leaf-feeding herbivores and mimicked the release of five commonly emitted volatiles individually. Three compounds (cis-3-hexen-1-ol, linalool, and cis-alpha-bergamotene) increased egg predation rates by a generalist predator; linalool and the complete blend decreased lepidopteran oviposition rates.

Nojima et al. (J Chem Ecol. February 2003; 29(2):321-36) used electroantennographic detection (GC-EAD) to identify volatile compounds from hawthorn fruit (Crataegus spp.) acting as behavioral attractants for hawthorn-infesting Rhagoletis pomonella flies. Consistent EAD activity was obtained for six chemicals: ethyl acetate (94.3%), 3-methylbutan-1-ol (4.0%), isoamyl acetate (1.5%), 4,8-dimethyl-1,3(E),7-nonatriene (0.07%), butyl hexanoate (0.01%), and dihydro-beta-ionone (0.10%). In a flight-tunnel bioassay, there was a dose-related increase in the percentage of flies flying upwind to the six-component mixture.

The (17R)- and (17S)-isomers of volicitin contained in the oral secretion of the beet armyworm induces corn seedlings to emit a blend of volatile compounds to attract the natural enemy of the herbivore (Itoh et al., Biosci Biotechnol Biochem. July 2002;66(7):1591-6)

Chen et al. (Sci China C Life Sci. April 2004; 47(2):115-23) recorded electroantennogram (EAG) responses of male and female Oedipodinae grasshoppers, Oedaleus decorus asiaticus B.-Bienrlo and Angaracris barabensis Pall to 37 plant volatile compounds. Most of the green leaf volatiles elicited higher EAG responses in both species and sexes than terpenic compounds and some aromatic compounds. Strong EAG responses were elicited by 6-carbon alcohols (1-hexanol, Z-hexen-2-ol-1, E-2-hexen-1-ol, E-hexen-3-ol-1), aldehyde (E-2-hexen-1-al), ester (Z-hexen-3-yl acetate), and 7-carbon alcohols (1-heptanol) in both species and sexes.

Graus et al. (Plant Physiol. August 2004; 135(4):1967-75) investigated the release of acetaldehyde and other oxygenated volatile organic compounds (VOCs) from leaves of Grey poplar [Populus x canescens (Aiton) Smith] following light-dark transitions. Hexenal was emitted first, followed by acetaldehyde and other C(6)-VOCs.

The major VOCs released by Arabidopsis roots were either simple metabolites, ethanol, acetaldehyde, acetic acid, ethyl acetate, 2-butanone, 2,3,-butanedione, and acetone, or the monoterpene, 1,8-cineole (Steeghs et al., Plant Physiol. May 2004;135(1):47-58).

The process involved in the determination of chemical structure of volatile and semi-volatile chemicals involves, in general, the isolation of single chemical compounds from all other compounds; the collection of a range of instrumental data which allows deductions about the structure to be made; the purchase or synthesis (manufacture) of likely candidate compounds; and the demonstration that the natural material is identical in all respects with the compound purchased or synthesised.

With particular reference to plant chemistry, the process of isolation involves the trapping of volatile chemicals, the extraction of non-volatile chemicals, and the refining (separation) of the complex mixtures so obtained in some way so as to progressively home in on the sub-set of relevant chemicals. This process must involve the regular presentation of the natural material as it is refined to the insect under study to observe its behaviour. This bioassay-directed fractionation directs the course that the analytical process takes.

Separation and Isolation. The basic separation technique used by the organic chemists is chromatography—and it takes several forms depending very broadly on whether the chemicals being studied are volatile or non-volatile, and in the latter case water-soluble or not. The analytical instruments involved are called chromatographs. Thus, the analytical instrumentation will include Gas Chromatographs (GC) for the resolution and detection of volatile and semivolatile chemicals (though strictly speaking, the compounds being analyzed are rarely gases), and High Performance Liquid Chromatographs (HPLC) for the similar treatment of non volatile compounds. A variety of other related techniques can be applied.

Instrumental Data. The actual chemical structure determination of pure chemical compounds is not a trivial task, particularly if the compounds are unknown to science. Such compounds are often active towards insects at the level of a thousand-millionth of a gram, so the instruments which yield data that can be used to hypothesize structures are remarkably sensitive. In a sense, the scientist is hoping to bring a level of sensitivity to detection which is equivalent to the sensory apparatus of the insect itself. In fact, the insect antenna—in a process called electroantelnography—can be used to help identify those chemicals in a complex mixture of volatiles which are most likely to be influencing the insect's behaviour.

Instruments used include a variety of devices which fall within the categories of spectrometers and spectrophotometers. Modem instruments are sophisticated enough so that much information can be derived from a single instrument which combines the separation/isolation stage with the spectrometric stage.

Synthetic chemistry. The determination of the chemical structure will often involve various chemical manipulations of the natural material itself, as well as the actual synthesis of the target compound from synthetic or natural precursors. The demonstration of exact equivalence of the synthetic and natural product in all ways, from properties revealed by a range of instruments to properties demonstrated towards insects must be followed.

### IV. INSECT ODORANT RECEPTORS

Drosophila Odorant Receptors. The cellular basis of the odor code has been explored in detail in Drosophila, whose relatively simple olfactory system allows precise physiological measurements of individual ORNs in vivo. Flies contain two olfactory organs, the antenna and the maxillary palp, which contain ˜1200 and ˜120 ORNs, respectively (Stocker, 1994; Shanbhag, 1999, 2000). These ORNs are compartmentalized in olfactory sensilla, which divide into morphologically distinct classes, including large basiconic sensilla, small basiconic sensilla, trichoid sensilla, and coeloconic sensilla. Each sensillum contains up to four neurons, whose activities can be defined by extracellular electrophysiological recordings (Dobritsa, 2003).

In Drosophila, extensive recordings have revealed that ORNs fall into distinct functional classes based on their odor response spectra. Sixteen functional classes of ORNs, each with a unique response spectrum to a panel of 47 odors, were identified from recordings of antennal basiconic sensilla (De Bruyne, 2001). These ORNs exhibit diverse response dynamics, including excitatory and inhibitory responses, and various modes of termination kinetics. The 16 ORN classes are found in stereotyped combinations in seven functional types of basiconic sensilla, each mapping to a defined subregion of the antennal surface (Dobritsa, 2003).

Functional differences among ORN classes are believed to aiise from the expression of different odor receptors. A family of at least 60 seven-transmembrane-domain receptor genes, the Or genes, was discovered in Drosophila and proposed to encode odor receptors (Clyne, 1999a; Gao, 1999; Vosshall, 1999, 2000). Individual Or genes are expressed in different subsets of ORNs. A mutation that alters the expression of a subset of Or genes alters the odor specificity of a subset of ORNs (Clyne, 1999b), and direct evidence (Störtkuhl and Kettler, 2001; Wetzel, 1999), was recently found for the involvement of one Or gene in olfactory signaling (Dobritsa, 2003).

Or22a and Or22b, were the first Or genes identified in a computational screen for Drosophila odor receptors (Clyne et al., 1999a; U.S. Pat. No. 6,610,511, specifically incorporated by reference herein). These genes are tightly clustered, lying within 650 bp of each other in the genome. Clustering is common among Or genes, with more than one-third of the family members located in clusters of up to three genes. Or22a and Or22b are among the most closely related members of the family, showing 78% amino acid identity (Dobritsa, 2003).

Individual receptors are demonstrated to map to individual neuronal classes through a genetic and molecular analysis of the two Or genes, Or22a and Or22b. There are three functional types of large basiconic sensilla, ab1, ab2, and ab3, defined on the basis of electrophysiological recordings from the ORNs they contain. The sensilla expressing Or22a and Or22b contain an A neuron whose strongest responses are to ethyl butyrate, pentyl acetate, and ethyl acetate and a B neuron whose strongest responses are to heptanone, hexanol, and octenol. The Or22a receptor is shown to map to the ab3A neuron, by using the Or22a promoter and the GAL4-UAS system to drive expression of GFP or the cell death. gene reaper, followed by physiological recordings from individual sensilla. The Or22a receptor is thereby linked to the odor ethyl butyrate, to which ab3A is highly sensitive (Dobritsa, 2003).

Analysis of a mutant lacking Or22a, together with rescue experiments using an Or22a transgene, confirm the mapping of Or22a to the ab3A neuron. This genetic analysis provides direct evidence that an Or gene is required in vivo for normal odor detection (Dobritsa, 2003). The demonstration that deletion mutants lacking Or22a and Or22b are defective in odor response and that the response is restored upon introduction of an Or22a transgene provide direct evidence that Drosophila Or genes are in fact critical components of olfactory signal transduction. The effect of the deletion mutation is specific: the mutation has a profound effect on the ab3A neuron but no other ORN among the large basiconic sensilla. The response of the ab3A cell is eliminated for all odors tested. The finding that deletion of Or22a and Or22b eliminates response to all tested odors, and that the full response spectrum can be rescued by Or22a alone, suggests that the broad response spectrum documented for ab3A can be attributed to one receptor, Or22a (Dobritsa, 2003).

Hallem et al. (Cell (2004) 117:965-979) subsequently undertook a functional analysis of the known receptor repertoire in the Drosophila antenna, thereby establishing a odor response spectrum.

Mosquito Odorant Receptors. The human body surface excretes many compound types, a proportion of which contribute to overall human volatile emissions (Sastry et al., 1980). The excreted compounds are modified through microbial action, particularly on the essentially odorless sweat secreted from the apocrine glands situated in the axillary regions (e.g. armpits) (Stoddart, 1990). Female mosquitoes detect the presence of these compounds by the odorant receptors (ORs) located on their antennae and initiate an olfactory signal transduction cascade. Anopheles include several species of mosquitoes that transmit numerous parasitic and viral diseases, including malaria, dengue, West Nile encephalitis, and yellow fever. In the malaria vector mosquito, Anopheles gambiae, olfaction plays a major role in establishing host preference and blood feeding behavior for disease transmission.

Five of these odorant receptor genes, AgOr1, AgOr2, AgOr3, AgOr4 and AgOr5, that encode candidate odorant receptor proteins from A. gambiae have been identified and characterized using genomics and molecular-based approaches (see, e.g., U.S. Pat. Appl. Pub. No. 2003/0166013, which is incorporated in its entirety herein). A family of odorant receptors is found on the third chromosome of Anopheles gambiae. To date, a total of 79 odorant receptor (AgOR) genes have been identified and characterized. in A. gambiae (Hill et al., (2002) Science 298: 176-178.).

AgOr1 is expressed specifically in the olfactory tissue of female but not male mosquitoes, and its expression is down-regulated following a blood meal (Fox et al., 2001); host-seeking behavior of these mosquitoes is also female-specific and reduced by blood-feeding (Takken, 2001). AgOr1 is expressed specifically in the olfactory tissue of female mosquitoes but not male mosquitoes. It is a component of a specific olfactory signal transduction cascade that is active before blood feeding in A. gambiae adult females and its expression is down-regulated following a blood meal suggesting that AgOr1 plays a critical role in establishing the host preference that is a central element in A. gambiae high overall capacity to transmit malaria (Fox et al., 2001; Fox et al., 2002).

Moth Odorant Receptors. Rostelien et al. (J Comp Physiol [A]. September 2000;186(9):833-47) identified the selective receptor neurone responses to E-beta-ocimene, beta-myrcene, E,E-alpha-famesene and homo-famesene in the moth Heliothis virescens by using gas chromatography linked to electrophysiology.

A divergent gene family encoding candidate olfactory receptors of the moth H. virescens was identified by Krieger et al. (Eur J Neurosci. August 2002;16(4):619-28). Candidate olfactory receptors of the H. virescens were found to be extremely diverse from receptors of the fruitfly D. melanogaster and the mosquito A. gambiae, but there is one exception. The moth receptor type HR2 shares a rather high degree of sequence identity with one olfactory receptor type both from Drosophila (Dor83b) and from Anopheles (AgamGPRor7).

Krieger et al. (J Comp Physiol A Neuroethol Sens Neural Behav Physiol. July 2003;189(7):519-26) identified HR2 homologues in two further lepidopteran species, the moths Antheraea pernzyi and Bombyx mori, which share 86-88% of their amino acids.

Silkworm Odorant Receptors. Screening of antennal cDNA libraries with an oligonucleotide probe corresponding to the N-terminal end of a Antheraea polyphemus pheromone-binding protein (PBP), led to the identification of fall length clones encoding this protein in the sillkworm species A. polyphemus (Apol PBP3) and A. pernyi (Aper PBP3) (Kreiger et al., Eur J Biochem. May 2000;267(10):2899-908). By screening the antennal cDNA library of A. polyphemus with a digoxigenin-labelled A. pernyi PBP2 cDNA (Krieger et al. (1991) Biochim. Biophys. Acta 1088, 277-284) a homologous PBP (Apol PBP2) was cloned. Binding studies with the two main pheromone components of A. polyphemus and A. pernyi, the (E,Z)-6,11-hexadecadienyl acetate (AC1) and the (E,Z)-6,11-hexadecadienal (ALD), revealed that in A. polyphemus both Apol PBP1a and the new Apol PBP3 bound the 3H-labeled acetate, whereas no binding of the 3H-labeled aldehyde was found. In A. pernyi two PBPs from sensory hair homogenates showed binding affinity for the AC1 (Aper PBP1) and the ALD (Aper PBP2), respectively.

Tanoue et al. (Insect Biochem Mol Biol. September 2001;31(10):971-9) reported the isolation of cDNA of the receptor type GC, designated BmGC-I, from the male silkmoth antennae. The deduced amino acid sequence indicates that BmGC-I appears to consist of four domains: an extracellular, single transmembrane, kinase-like and a guanylyl cyclase domain. BmGC-I is most closely related to the mammalian natriuretic peptide hormone receptor A (GC-A) and retains all the cysteine residues that are conserved within the extracellular domain of the mammalian GC-As.

### V. ORGAMISM DETECTION, MONITORING, AND CONTROL

General Pest Management. The present invention can be used to identify and modify compounds which may be used for pest management. It is especially desirable to utilize various aspects of the present invention for pest management related to crop protection.

The application of pheromones is now firmly established as a key component of pest management and control, especially within the framework of integrated pest management (IPM). An object of organism control is to modulate an organism's behavior or activity so as to reduce the irritation, sickness, or death of the host (e.g., a plant host), or to decrease the general health and proliferation of the organism.

For example, the propagation of a mouse population in a given area of actual or potential mice infestation may be prevented or inhibited by treating such an area with an effective amount of male mouse pheromones, wherein such pheromones have male mouse aversion signaling properties (see, e.g., U.S. Pat. No. 5,252,326).

Insect Repellents and Insecticides. The present invention provides the tools and methodologies useful for identifying compounds which modulate insect behavior by exploiting the sensory capabilities of the target insect. For example, attempts have been made to describe and synthesize the complex interactions which underlie host-seeking behavior in mosquitoes. Using the methods and olfactory receptor genes of the present invention, it is possible to design specific compounds which target mosquito olfactory receptor genes. Thus, the present invention provides the ability to alter or to eliminate the orientation and feeding behaviors of mosquitoes and thereby have a positive impact on world health by controlling mosquito-borne diseases, such as malaria.

Olfactory receptor genes may be targeted using various aspects of the present invention. For example, based on the present invention probes can be designed for the identification and characterization of new olfactory receptor genes. Putative olfactory receptor genes can be isolated from various insect species and utilized in the various screening methods described elsewhere herein, such as the high throughput assays to identify synthetic and natural compounds which may modulate the behavior of the insect.

Mating Enhancement and Disruption. The olfactory receptor genes studied as discussed herein may be used to identify compounds which interfere with the orientation and mating of a wide range of organisms, including insects. Thus, the present invention enables the identification of compositions which disrupt insect mating by selective inhibition of specific receptor genes involved in mating attraction (see, e.g., U.S. Pat. No. 5,064,820).

Animal Repellants. The identification of receptors for odorants may be useful in developing new insect repellants and traps for the control of mosquitoes and other insect pests. The olfactory receptor genes studied using the materials, systems and methods of the present invention may be used to identify compounds which can be used as animal repellants. Such compositions may be used to repel both predatory and non-predatory animals (see, e.g., U.S. Pat. No. 4,668,455).

For example, useful toxicants (or insecticides) for yellowjackets include organophosphorous toxicants, carbamates, inorganic toxicants and insect growth regulators. Specific compounds include dimethyl (2,2,2-trichloro-1-hydroxyethyl) phosphonate, 2,2-dichlorovinyl dimethyl phosphate and 1,2-dibromo-2,2-dichloroethyl dimethyl phosphate. Poisoned baits are provided by combining, mixing or blending toxicant with a feeding stimulant such as pet food.

Controlled release devices which gradually release pesticides can be constructed to prevent, for a prolonged period of time, intrusions by insects into areas, structures or objects that are sought to be protected from intrusions. Any polymer which can provide the desired release rate and which does not destroy the pesticidal nature of the pesticide used in the device can be employed to provide a polymeric. Generally, suitable polymers can include both thermoset and thermoplastic polymers. Currently preferred polymers are silicones, urethanes, polyurethanes, acrylonitrile butadiene, acrylic rubber, styrene-vinyl rubber EVA and polyethylenes. Especially preferred are the following polymers: RTV-41, Hytrel, Solithane, Nipol 1312, Nipol 1312 LV, Hycar X16, Kraton D1101, Ultra Clear, Aromatic 80A urethane, Pellethane 2102-80A, Pellethane 2102-55D Alipmtic PS-49-100, Polyurethane 3100, Polyurethane 2200, EVA 763, Polyethylene MA 7800, and Polyethylene MA 78000. In some control release devices of the present invention a carrier can be included to produce a desired release rate. A carrier can be carbon black clay or amorphous silica.

The protection against intrusion is provided by the present invention as the result of the accumulation of the pesticide on the surface of the polymer matrix and/or the accumulation of the pesticide in an absorbent medium in contact with or in close proximity to the matrix, when the insect or other cold blooded animal comes in contact with pesticide it is repelled by it and/or killed by it. In case of insects, the pesticide is generally transferred to the feet of the insects and when the release rate of the pesticide is at least about 10 .mu.g/cm2/day, sufficient amount of pesticide adheres to insect to kill it. It has been discovered that faster release rates are necessary for larger cold blooded animals. For snakes, and other cold blooded vertebrae animals, the pesticide release rates must be at least 40 .mu.g/cm2/day.

Animal Attractants. The olfactory receptor genes studied using the materials, systems and methods of the present invention may be used to identify compounds which attract specific insects to a particular location (see, e.g., U.S. Pat. Nos. 4,880,624 & 4,851,218). For example, attracting the predators and parasitoids which attack certain pests offers an alternative method of pest management.

Aspects of the present invention may to used in various methods which reduce or eliminate the levels of particular insect pests, such as mosquitoes and tsetse flies. Traps may also be utilized where trapped insects are killed by toxicant-containing poison baits where the yellowjackets may consume poisoned bait. As a particular example, insect traps can be created wherein the pheromone attracts a particular insect, like the tsetse fly, and the insect so attracted dies in the trap. In this way, the population of tsetse flies may be reduced or eliminated in a particular area. For example, 4-methylphenol has been shown to increase the effectiveness of traps for the tsetse fly, Glossina morsitans morsitans (Vale et al., 1988).

The insect attractant compositions so identified may also be combined with an insecticide, for example as an insect bait in microencapsulated form. Alternatively, or in addition, the insect attractant composition may be placed inside an insect trap, or in the vicinity of the entrance to an insect trap.

In addition to killing insects, the trapping of insects is often very important for estimating or calculating how many insects of a particular type are feeding within a specific area. Such estimates are used to determine where and when insecticide spraying should be commenced and terminated.

Attractants, dispensers and/or lures are useful in combination with traps. An effective trapping system includes a trapping means and a dispenser means located within the trapping means which provides an effective attractant amount of a vapor blend of the vapors of components (A) and (B). A trapping means is any device for catching insects, particularly yellowjackets and includes, but is not limited to, a number of traps which are commercially available [sticky-wing traps (TackTrap™, Animal Repellants, Inc., Griffin, Ga.); Yellowjacket Trappit Dome traps, Agrisense, Fresno, Calif.; water traps (Rescue!®., Sterling International, Inc., Spokane, Wash.)] and which are described in the art (e.g. U.S. Pat. Nos. 5,557,880; 5,522,172; 5,501,033; 5,339,563). A preferred trap has a mixing chamber where vapors of components (A) and (B) form a blend and the vapor blend exits the trap chamber to attract yellowjackets to a chamber from which they cannot escape. The attractant components that produce the attractant vapor blend may be present as a mixture or in separate dispensers within the trap. The components also may be added directly to a drowning solution that can be used in the trap, with the vapor blend emanating from the drowning solution. Additives may be present in the drowning solution that aid in the capture and killing of attracted yellowjackets, such as detergents or wetting agents, clays, dyes and toxicants, as long as the additives do not substantially interfere with the effectiveness of the attractant blend.

Insect traps which may be used are, for example, those as described in PCT/BG93/01442 and U.S. Pat. No. 5,713,153. Specific examples of insect traps include, but are not limited to, the Gypsy Moth Delta Trap®, Boll Weevil Scout Trap®, Jackson trap, Japanese beetle trap, McPhail trap, Pherocon 1C trap, Pherocon II trap, Perocon AM trap and Trogo trap.

Kairomones may be used as an attractancy for the enhancement of the pollination of selected plant species.

Attractant compositions which demonstrate biological activity toward one sex which is greater than toward the opposite sex may be useful in trapping one sex of a specific organism over another. For example, a composition may be a highly effective attractant for male apple ermine moths (Yponomeuta malinellus (Zeller)) and not so effective an attractant for female apple ermine moths. By attracting adult males to field traps, the composition provides a means for detecting, monitoring, and controlling this agricultural pest (see, e.g., U.S. Pat. No. 5,380,524).

A pheromone-containing litter preparation may attract the animals and absorb liquids and liquid-containing waste released by the attracted animal (see, e.g., U.S. Pat. No. 5,415,131).

Eddy et al. (U.S. Pat. No. 3,912,810) describe a method of attracting yellowjackets using an ester having an alcohol and an acid moiety and having a chain length of from 10 to 12 carbon atoms. 2,4-Hexadienyl butyrate, 2,4-hexadienyl propionate and 2,4-hexadienyl isobutyrate were shown to attract V. pensylvanica (Davis et al. 1967. J. Med. Entomol. vol. 4, pp. 275-280) as well as heptyl butyrate (Davis et al. 1969. J. Econ. Entomol. vol. 62, p. 1245; Davis et al. 1973. Environmental Entomol. vol. 2, pp. 569-571; MacDonald et al. 1973. Environmental Entomol. vol. 2, pp. 375-379) and octyl butyrate (Davis et al. 1972. Environmental Entomol. vol. 1, p. 673; McGovern et al. 1970. J. Econ. Entomol. vol. 63, pp. 1534-1536).

Chemical attractants for yellowjackets and wasps have also been described by Landolt, P. J. (1998. Environmental Entomol. vol. 27, no. 4; Landolt, P. J., U.S. Pat. No. 6,083,498, 2000) and include compositions of vapor blends of acetic acid and one or more of isobutanol, racemic 2-methyl-1-butanol, S-(−)-2-methyl-1-butanol, 2-methyl-2-propanol, heptyl butyrate and butyl butyrate. Aldrich et al. (1986. Experientia. vol. 42, pp. 583-585) identified components of the pheromone of the predaceous spined soldier bug which attract eastern yellowjacket (and related species) workers and queens: mixtures of either (E)-2-hexenal and .alpha.-terpineol or (E)-2-hexenal and linalool.

In addition, various yellowjacket traps are commercially available which require baits based on sugar or pet food meat products. A combination of volatile components which act synergistically to effectively attract yellowjackets in the Vespula species group includes (E)-2-hexenal and linalool in the first component (A) and acetic acid and isobutanol in the second component (B) (U.S. Pat. No. 6,740,319).

### VI. TRANSGENIC ORGANISMS

Transgenic insects containing mutant, knock-out or modified genes can be produced using various methods known in the art. Transgenic insects are genetically modified insects into which recombinant, exogenous or cloned genetic material has been experimentally transferred. Such genetic material is often referred to as a “transgene”. The nucleic acid sequence of the transgene may be integrated either at a locus of a genome where that particular nucleic acid sequence is not otherwise normally found or at the normal locus for the transgene. The transgene may consist of nucleic acid sequences derived from the genome of the same species or of a different species than the species of the target insect.

The term a “germ cell line transgenic insect” refers to a transgenic insect in which the genetic alteration or genetic information was introduced into a germ line cell, thereby conferring the ability of the transgenic insect to transfer the genetic information to offspring. If such offspring in fact possess some or all of that alteration or genetic information, then they too are transgenic insects.

The alteration or genetic information may be foreign to the species of insect to which the recipient belongs, foreign only to the particular individual recipient, or may be genetic information already possessed by the recipient. In the last case, the altered or introduced gene may be expressed (i.e., over-expression and knock-out) differently than the native gene. Transgenic insects can be produced by a variety of different methods including P element-mediated transformation by microinjection (see, e.g., Rubin & Spradling, (1982) Science 218, 348-353; Orr & Sohal, (1993) Arch. Biochem. Biophys. 301, 34-40), transformation by microinjection followed by transgene mobilization (Mockett et al., (1999) Arch. Biochem. Biophys. 371, 260-269), electroporation (Huynh & Zieler, (1999) J. Mol. Biol. 288, 13-20) and through the use of baculovirus (Yamao et al., (1999) Genes Dev. 13, 511-516). Furthermore, the use of adenoviral vectors to direct expression of a foreign gene to olfactory neuronal cells can also be used to generate transgenic insects (see, e.g., Holtmaat et al., (1996) Brain. Res. Mol. Brain Res. 41, 148-156).

A number of recombinant or transgenic insects have been produced, including those which over-express superoxide dismutase (Mockett et al., (1999) Arch. Biochem. Biophys. 371, 260-269); express Syrian hamster prion protein (Raeber et al., (1995) Mech. Dev. 51, 317-327); express cell-cycle inhibitory peptide aptamers (Kolonin & Finley (1998) Proc. Natl. Acad. Sci. USA 95, 14266-14271); and those which lack expression of the putative ribosomal protein S3A gene (Reynaud et al., (1997) Mol. Gen. Genet. 256, 462-467).

While insects remain the preferred choice for most transgenic experimentation, in some instances it is preferable or even necessary to use alternative animal species. Transgenic procedures have been successfully utilized in a variety of animals, including mice, rats, sheep, goats, pigs, dogs, cats, monkeys, chimpanzees, hamsters, rabbits, cows and guinea pigs (see, e.g., Kim et al, (1997) Mol. Reprod. Dev. 46, 515-526; Houdebine, (1995) Reprod. Nutr. Dev. 35, 609-617; Petters, (1994) Reprod. Fertil. Dev. 6, 643-645; Schnieke et al., (1997) Science 278, 2130-2133; and Amoah, (1997) J. Anim. Sci. 75, 578-585).

The method of introduction of nucleic acid fragments into insect cells can be by any method which favors co-transformation of multiple nucleic acid molecules. For instance, Drosophila embryonic Schneider line 2 (S2) cells can be stably transfected as previously described (Schneider, (1972) J. Embryol. Exp. Morphol. 27, 353-365). Detailed procedures for producing transgenic insects are readily available to one skilled in the art (see Rubin & Spradling, (1982) Science 218, 348-353; Orr & Sohal, (1993) Arch. Biochem. Biophys. 301, 34-40, herein incorporated by reference in their entirety).

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### III. EXAMPLES

### Example 1

### Production of Transgenic ab3a Mutant Drosophila Lines

A mutant ab3A antennal neuron (Δab3A) lacks odor response due to the deletion of its endogenous receptor genes, Or22a and Or22b. The ab3A mutant flies lacking both Or22a and Or22b genes are referred to as Δhalo.

To construct the synthetic deficiency Δhalo, deletion Df(2L)dp79b (break points 22A2-3; 22D5-E1) and the duplication Dp(2;2)dppd21 (break points 22A2-3; 22F1-2; inserted at 52F) were combined onto a single chromosome. (Fly stocks, mutant chromosomes, and break-point data for deletions are described on FlyBase (http://flybase.bio.indiana.edu).

A 8.2 kb region upstream of the Or22a translational start codon was isolated and fused to the coding sequence of GAL4 to generate a construct 22a-GAL. Flies carrying the 22a-GAL4 transgene were crossed to flies carrying UAS-GFP to yield progeny in which GAL4 binds to a UAS and activates transcription of GFP. The GFP-labeled sensilla are visible in live animals, thereby allowing us to distinguish them and record electrophysiological responses to various odors (Dobritsa, 2003).

In summary, odorant receptors are introduced specifically into Δab3A using the GAL4/UAS system and Or22a-GAL4 is used to drive expression from a UAS-Or construct. The odorant response of the neuron (Δab3A:OrX) is assayed electrophysiologically.

The response of the Drosophila cell could be examined by measuring either the action potential response or the receptor potential response.

### Example 2

### Preparation of AgOr1 and AgOr2 cDNA Clones

A. gambiae sensu stricto (G3 strain) embryos (provided by Mark Benedict, Centers for Disease Control and Prevention, Atlanta, Ga.) were disinfected with 0.05% sodium hypochlorite before hatching in flat plastic pans with distilled water. Larvae were reared on a diet of ground Whiskas® Original Recipe cat food (KalKan, Vernon, Calif.), which was applied to the surface of the water. Pupae were transferred to plastic cups in one-gallon (approx. 4 liters) plastic containers, where newly emerged adults were collected the next morning. Adult mosquitoes were maintained in one-gallon plastic containers at 27° C. with 75% relative humidity under a 12:12 h photoperiod and provided a 10% dextrose solution.

Antennae/maxillary palps (oltactory tissues) were hand dissected from 4-day old adult mosquitoes. These tissues were used to generate RNA and, subsequently, cDNA template pools for PCR. Furthermore, as an additional control, all reactions were carried out using oligonucleotide primers that were designed to span predicted introns in order to distinguish between genomic DNA and cDNA templates, as well as oligonucleotide primers against the A. gambiae ribosomal protein S7 (rps7) (Salazar et al., 1993, Nucleic Acids Res. 21: 4147). The rps7 gene is constitutively expressed at high levels in all tissues of the mosquito and, therefore, provides a control for the integrity of the cDNA templates. The RT-PCR products from AgOR1 and AgOr2 were subcloned and sequenced.

The GenBank accession numbers for the AgOr1 and AgOr2 cDNA sequences used in these experiments are AF364130 and AF364131, respectively. See also Fox et al, 2001 PNAS.

### Example 3

### Expression of Mosquito Odorant Receptors AgOr1 and AgOr2 in a Drosophila Olfactory Receptor Neuron

To investigate the function of the A. gambiae odorant receptor genes AgOr1 and AgOr2 directly, these genes were expressed in the Drosophila olfactory receptor neuron that lacks odor response due to the deletion of its endogenous receptor genes, Or22a and Or22b using an Or22a promoter and the GAL4-UAS system.

AgOr1 and AgOr2 were expressed in the Δhalo background using the GAL4-UAS system. Specifically AgOr1 or AgOr2 was placed under the control of a UAS and expressed using the 22a-GAL4 driver so as to drive its expression in ab3A cells. The genotypes of the transgenic flies expressing AgOr1 and AgOr2 genes were UAS-AgOr1/Or22apromoter-Gal4 and UAS-AgOr2/Or22aproiiioter-Gal4.

### Example 4

### Identification of a Mosquito Odorant Receptors that Respond to a Components of Human Sweat

To identify compounds to which the mosquito Odorant receptors AgOr1 and AgOr2 respond, the odor response of the Drosophila olfactory receptor neuron, ab3A, was assayed by single-unit electrophysiology. The chemicals tested in the assay were ethyl acetate, pentyl acetate, ethyl butyrate, methyl salicylate, 1-hexanol, 1-octen-3-ol, E2-hexenal, 2-heptanone, geranyl acetate, CO2, paraffin oil, 4-methylphenol, 2-methylphenol, 3-methylphenol, 4-propylphenol, 4-ethylphenol, benzaldehyde, benzyl alcohol, benzyl acetate, 4-isopropylbenzaldehyde, cyclohexanone, cyclohexanol, 4-methylcyclohexanol and 2,3-dibutanone. Transgenic flies of the genotype UAS-Or22apromoter-Gal4, UAS-AgOr1/Or22apromoter-Gal4and UAS-AgOr2/Or22apromoter-Gal4 were used in the study.

Action potentials of the ORNs in a sensillum were recorded by placing an electrode through the sensillum wall into contact with the lymph that bathes the dendrites. Drosophila males aged <1 week were mounted as in Clyne et al. (1997) and De Brnyne et al. (2001). The antennal surface was observed at 1200× magnification, which allowed individual sensilla to be clearly resolved, through an Olympus BX40 microscope fitted with fluorescence optics to view GFP. For the recording electrode, a glass capillary with the tip drawn to <1 μm diameter was filled with sensillum lymph ringer (Kaissling and Thorson, 1980) and slipped over an AgCl-coated silver wire. The indifferent electrode was filled with Ephrussi and Beadle solution (Ashburner, 1989) and was put into the head. Signal from the recording electrode was led into a >10¹²Ω input impedance amplifier (IsoDam, WPI, Sarasota, Fla.), fed through a 100 Hz high-pass filter into an AD-interface (GW Instruments, Somerville, Mass.). Recordings were analyzed offline in IGOR-Pro (WaveMetrics, Lake Oswego, Oreg.).

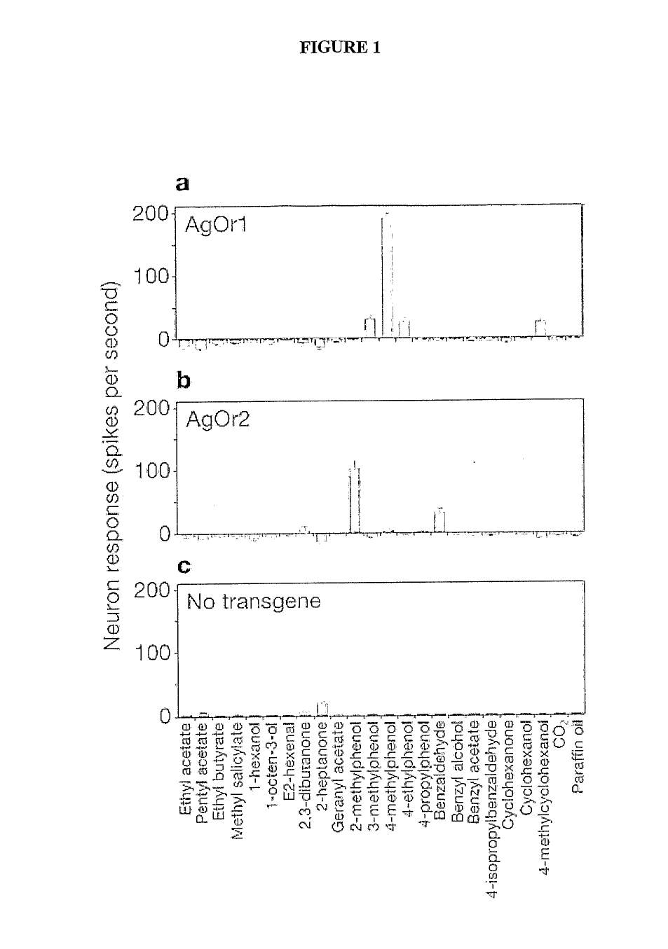

Odor stimuli were presented from Pasteur pipettes holding solutions of chemicals in paraffin oil on filter paper. Liquid odors were diluted 10⁻⁴ in paraffin oil and solid odors were diluted 0.2 mg/ml in paraffin oil whereupon an aliquot of 50 μl was dropped on a 0.5 inch filterroundel placed in the shaft of a Pasteur pipette. A pipette with CO2 was prepared by displacing the air with CO2 from a tank. Stimuli were presented by placing the tip of a Pasteur pipette through a hole in a tube that carried an air stream (37.5 ml/s) over the fly and redirecting a flow of N2 (3.75 ml/s) by solenoid-control through the pipette to give a 0.5 s pulse. Fresh stimulus pipettes were prepared after a maximum of three presentations; the CO2 cartridge was renewed after single use. Responses of the ab3A neurons were quantified by subtracting the number of impulses (i.e., spikes) in 500 ms of spontaneous activity from the number in the 500 ms after the onset of odor stimulation (FIG. 1).

Recordings from labeled sensilla of mutant flies (control), containing a olfactory neuron carrying a deletion of its endogenous receptor genes, Or22a and Or22b, revealed that the ab3A neurons are unresponsive to all odors of the test panel: for all odors tested, the mean response is <=18 spikes/sec (FIG. 1, c). Odor response spectrum of the mutant flies expressing AgOr1 showed a strong response to the odorant 4-methylphenol (FIG. 1, a). The mutant flies expressing AgOr2 found a different odor response spectrum. In contrast to AgOr1, AgOr2 confers a strong response to 2-methylphenol, but not 4-methylphenol (FIG. 1, b).

These results demonstrate that AgOr genes encode odorant receptors, and that the female-specific receptor AgOr1 plays a role in the host-seeking behavior of A. gambiae. The ability of mosquito odorant receptors to function in Drosophila, in the absence of other mosquito-specific proteins, suggests a broad and unexpected compatibility between odorant receptors and olfactory receptor neurons of different species, and demonstrates the utility of the fruit fly as an in vivo model system for the study of odorant receptors derived from less genetically tractable insect species. The identification of receptors for particular human odorants suggests their use in screening for ligands that activate or inhibit these receptors. Some such ligands may be useful in traps, and others may be useful as repellents.

### Example 5

### Expression of Putative Odorant Receptors From European Corn Borers

The European corn borer, Ostrinia nubilalis (Hubner) (Lepidoptera: Crambidae), is a major pest of maize, Zea mays L., in many temperate parts of the world. Odorant receptor genes of O. nubilalis can be identified by using the algorithm disclosed in U.S. Pat. No. 6,610,511 and/or by selecting putative receptor genes with high sequence homology to the known odorant receptor genes identified in Drosophila, mosquitoes and silkworms as discussed above.

Once the putative odorant receptor genes are identified by one or more of these methods, the genomic DNAs and the corresponding cDNAs can be isolated using standard nucleic acid isolation techniques well known to one skilled in the gene isolation art. See, for example, U.S. Pat. No. 6,610,511, which discusses the identification and isolation of odorant receptors and is incorporated herein in its entirety.

The isolated genomic DNAs or the cDNAs of the putative odorant receptors can be tested against various chemicals using the UAS-Or/Or22apromoter-Gal4 system as set forth above.

One testing method could include testing the responses of the putative odorant receptors to various volatile and semi-volatile chemicals emitted by corn plants to determine whether any of these compounds bind with the receptor so as to produce firing of the Aab3A neuron. The identitfication of such compounds could lead to the design of effective corn border traps and/or methods of blocking the receptors.

Alternatively, another testing method could include testing responses of the putative odorant receptors to various volatile and semi-volatile chemicals known or suspected to repel, injure, or kill European corn borers. The identification of such compounds could lead to the selection of good candidates for the further study and development of chemicals to control the corn borers on a corn plant and elsewhere.

### Example 6

### In Vivo Odorant Receptor Systems for High Throughput Screening

The in vivo odorant receptor systems of the present invention can be used to screen numerous chemicals sequentially or for screening mixtures of chemicals.

For either method, extracellular single-unit recordings are performed essentially as described previously by de Bruyne et al. (2001, Neuron 30:537-552). Odorant stimuli and CO₂ stimuli are prepared in Pasteur pipettes as described previously (Dobritsa et al., 2003).

Chemicals are >99% pure or of the highest purity available (e.g., Fluka®, Sigma®, and Aldrich®), preferably racemic mixtures and usually diluted 10⁻² in H₂O. Solid odorants are dissolved 0.1 g in 5 ml paraffin oil.

Stimuli are presented by placing the tip of the pipette through a hole in a tube carrying a purified air stream (24 ml/s) directed at the fly and administering a 0.5 s pulse of charcoal-filtered air (5.9 ml/s) through the pipette containing the odorant. CO₂ stimuli are used once; all other stimuli are used for a maximum of three presentations.

Excitation are quantified from a count of the number of impulses during the 0.5 s stimulus period and/or by subtracting the number of impulses in the 1 s prior to odorant stimulation from the number of impulses in the 1 s following odorant stimulation. Each recording will be from a separate sensillum, with no more than 3 sensilla analyzed per fly.

EPGs are obtained as described previously by Ayer et al. (1992, J. Neurobiol. 23:965-982). Odor stimuli are prepared as for single-unit recordings, except that the pipette is connected by ˜2.5 cm of plastic tubing to a 5 ml syringe. Stimuli are presented by placing the tip of the pipette through a hole in a tube carrying a charcoal-filtered air stream (˜2 l/min) over the fly, and rapidly depressing the plunger of the syringe so as to pass 3 ml of air through the pipette and into the air stream. Recordings are obtained from flies aged <3 weeks. Error bars represent SEM.

For testing a succession ot individual chemicals using the same neuron, each successive chemical is introduced as a 0.5 s puff of odor as described above. Generally, it is sufficient to wait about 30 seconds between each puff of odor, although a waiting period of about 1 minute may be necessary if the chemical causes good firing of the neuron. One can easily determine when it is appropriate to introduce the next puff of odor by waiting until the neuron firing returns to base line firing before introducing the next puff of odor.

Alternatively, one can test mixtures of more than one chemical at a time using the in vivo odorant receptor systems of the present invention. In this way, it is possible to test a single puff of “odor” wherein the odor consists of any number of different chemical entities, such as a mixture of 2 chemicals, about 5 chemicals, about 10 chemicals, about 15 chemicals, about 20 chemicals, about 25 chemicals, about 50 chemicals, about 100 chemicals, or any other number of chemicals. If the neuron fires when the mixture is introduced, then it means that one or more of the chemicals in the mixture bound with the odorant receptor. Further testing of the individual chemicals in the mixture, or various combinations of the individual chemicals in the mixture, will enable one to identify the chemical(s) in the mixture responsible for the neuron firing.

The foregoing detailed description has been given for clearness of understanding only and no unnecessary limitations should be understood therefrom as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### REFERENCES

1. Buck, L. and Axel, R. (1991) “A novel mulitgene family may encode odorant receptors: a molecular basis for odor recognition” Cell 65:175-187
2. Clyne, P. J., Warr, C. G., Freeman, M. R., Lessing, D., Kim, J. and Carlson, J. R. (1999) “A novel family of divergent seven-transmembrane proteins: candidate odorant receptors in Drosophila” Neuron 22: 327-338
3. Cork, A. & Park, K. C. Med Vet Entomol 10, 269-76 (1996).
4. Dobritsa, A. A., van der Goes van Naters, W., Warr, C. G., Steinbrecht, R. A. & Carlson, J. R. Neuron 37, 827-41 (2003).
5. Fox A N, Pitts R J, Zwiebel L J. (2002) “A cluster of candidate odorant receptors from the malaria vector mosquito, Anopheles gambiae” Chem Senses. 27(5):453-459
6. Fox A N, Pitts R J, Robertson H M, Carlson J R, Zwiebel L J.(2001) “Candidate odorant receptors from the malaria vector mosquito Anopheles gambiae and evidence of down-regulation in response to blood feeding” Proc Natl Acad Sci USA 98(25):14693-14697. Epub Nov. 27, 2001.
7. Gao, Q. and Chess, A. (1999) “Identification of candidate Drosophila olfactory receptors from genomic DNA sequence” Genoinics 60: 31-39
8. Gross, S. P., Guo, Y., Martinez, J. E. & Welte, M. A. Current Biology 13, 1660-1668 (2003).
9. Hallem E A, Nicole Fox A, Zwiebel L J, Carlson J R. “Olfaction: mosquito receptor for human-sweat odorant” Nature 427(6971), 212-213 (2004).
10. Hildebrand, J. G. & Shepherd, G. M. (1997) Annu. Rev. Neurosci. 20, 595-631
11. Hill, C. A., Fox, A. N., Pitts, R. J., Kent, L. B., Tan, P. L., Chrystal, M. A., Cravchik, A., Collins, F. H., Robertson, H. M., Zwiebel, L. J. Science 298, 176-178 (2002).
12. Mombaerts, P. (1999) Annu. Rev. Neurosci. 22, 487-509
13. Ngai, J., Dowling, M. M., Buck, L., Axel, R. and Chess, A. (1993) “The family of genes encoding odorant receptors in the channel catfish” Cell 72: 657-666
14. Pilpel, Y., Sosinsky, A. & Lancet, D. (1998) Essays Biochem. 33, 93-104
15. Storkuhl, K. F. and Kettler, R. (2001) “Functional analysis of an olfactory receptor in Drosophila melanogaster” Proc. Natl Acad. Sci. USA 98: 9381-9385.
16. Takken, W. in Olfaction in Mosquito-Hose Interactions (eds. Bock, G. R. & Cardew, G.) 302-312 (Wiley, New York, 1996).
17. Takken, W., van Loon, J. J. & Adam, W. J Insect Physiol 47, 303-310 (2001).
18. Takken, W. (1991) Insect Sci. Appl. 12, 287-295
19. Troemel, E. R., Chou, J. H., Dwyer, N. D., Colbert, H. A. and Bargmann, C. I. (1995) “Divergent seven transmembrane receptors are candidate chemosensory receptors in C. elegans” Cell 83: 207-218
20. Vale, G. A., Hall, D. R. & Gouch, A. J. E. (1988) Bulletin of Entomological Research 78: 293-300.
21. Vosshall, L. B., Amrein, H., Morozov, P. S., Rzhetsky, A. and Axel, R. (1999) “A spatial map of olfactory receptor expression in the Drosophila antenna” Cell 96: 725-736
22. Wetzel, C. H., Oles, M., Wellerdieck, C., Kuczkowiak, M., Gisselmann, G. and Hatt, H. (1999) “Specificity and sensitivity of a human olfactory receptor fumctionally expressed in human embryonic kidney 293 cells and Xenopus laevis oocytes” J. Neurosci. 19: 7426-7433
23. Wetzel, C. H., Behrendt, H., Gisselmalm, G., Storkulil, K. F., Hovemann, B. and Hatt, H. (2001) “Functional expression and characterization of a Drosophila odorant receptor in a heterologous cell system” Proc. Natl Acad. Sci. USA 98: 9377-9380
24. Zhang, Y., Chou, J. H., Bradley, J., Bargmann, C. I. and Zilu, K. (1997) “The Caenorhabditis elegans seven-transmembrane protein ODR-10 functions as an odorant receptor in mammalian cells” Proc. Natl Acad. Sci. USA 94: 12162-12167
25. Zhao, H., Ivic, L., Otaki, J. M., Hashimoto, M., Mikoshiba, K. and Firestein, S. (1998) “Functional expression of a mammalian odorant receptor” Science 279: 237-242

1. A Drosophila cell comprising an olfactory receptor neuron containing a non-Drosophila odorant receptor in place of its endogenous odorant receptor(s).

2. An in vivo system for determining whether a non-Drosophila odorant receptor binds to a test chemical, said system comprising:
a. a Drosophila cell comprising an olfactory receptor neuron containing a non-Drosophila odorant receptor in place of its endogenous odorant receptor(s);
b. a test chemical;
c. a means of contacting the Drosophila cell with the test chemical; and
d. a means to measure the neuron response to the test chemical.

3. A method of determining whether an odorant receptor responds to a test chemical, said method comprising contacting a Drosophila cell with the test chemical, said cell comprising an olfactory receptor neuron containing a non-Drosophila odorant receptor in place of its endogenous odorant receptor(s), and measuring the odor response of the neuron, thereby determining whether the odorant receptor binds the test chemical.

4. The cell of claim 1, wherein the olfactory receptor neuron is a ab3A neuron.

5. The cell of claim 1, wherein a Drosophila fly comprises the cell of claim 1.

6. The cell of claim 1, wherein the endogenous odorant receptors are encoded by Or22a and Or22b.

7. The cell of claim 1, wherein the non-Drosophila odorant receptor is from an insect that is a human pest or a plant pest.

8. The cell of claim 1, wherein the non-Drosophila odorant receptor is isolated from an insect of the genus Anopheles.

9. The cell of claim 1, wherein the non-Drosophila odorant receptor is encoded by AgOr1 or AgOr2.

10. The cell of claim 1, wherein the non-Drosophila odorant receptor is encoded by cDNA.

11. The system of claim 2, wherein the test chemical is a volatile or semi-volatile chemical.

12. The system of claim 2, wherein the test chemical is a component of mammalian sweat.

13. The system of claim 2, wherein the odor response is measured by single-unit electrophysiology.

14. The cell of claim 1, wherein the gene encoding the non-Drosophila odorant receptor is operably linked to an Or22a promoter sequence.

15. The cell of claim 1, wherein the gene encoding the non-Drosophila odorant receptor is operably linked to a Gal4 sequence.

16. The cell of claim 1, wherein the gene encoding the non-Drosophila odorant receptor is operably linked to a UAS sequence.

17. The cell of claim 1, wherein the gene encoding the non-Drosophila odorant receptor is operably linked to an Or22a promoter sequence, a Gal4 sequence, and a UAS sequence.

18. An in vivo system for determining whether an odorant receptor binds to at least one chemical in a test mixture of two or more chemicals, said system comprising:
a. a Drosophila cell comprising an olfactory receptor neuron containing a non-Drosophila odorant receptor in place of its endogenous odorant receptor(s).
b. a test mixture comprising two or more different chemicals;
c. a means of contacting the Drosophila cell with the test mixture; and
d. a means to measure the odor response of the neuron.

19. A method of determining whether an odorant receptor binds to at least one chemical in a test mixture comprising two or more chemicals, said method comprising contacting a Drosophila cell with the test mixture, said cell comprising an olfactory receptor neuron containing a non-Drosophila odorant receptor in place of its endogenous odorant receptor(s); and measuring the response of the neuron, thereby determining if the odorant receptor binds to at least one chemical in the mixture.

20. The method of claim 19 further comprising testing the individual chemicals in the mixture, either individually or in various combinations, thereby determining which chemical(s) in the mixture bound with the receptor to cause neuron firing.
